(19) United States
(12) Patent Application Publication
(10) Pub. No.: US 2010/0190659 A1
(43) Pub. Date: Jul. 29, 2010

(54) Compositions and Methods for Diagnosing and Assessing Inflammatory Myopathies

(75) Inventor: Steven Greenberg, Newton, MA (US)

Correspondence Address: LAW OFFICE OF MICHAEL A. SANZO, LLC, 15400 CALHOUN DR., SUITE 125, ROCKVILLE, MD, 20855, US

(73) Assignee: THE BRIGHAM AND WOMEN'S HOSPITAL, INC., Boston, MA (US)

(21) Appl. No.: 12/668,424

(22) PCT Filed: Jul. 7, 2008
(86) PCT No.: PCT/US08/08333

§ 371 Date: Feb. 27, 2010

Related U.S. Application Data
(60) Provisional application No. 60/929,775, filed on Jul. 12, 2007.

(51) Int. Cl. C40B 40/06; G01N 33/50

(52) U.S. Cl. 506/16; 435/7.24

(57) ABSTRACT

The present invention is directed to assay methods for inflammatory myopathies and microarray plates that can be used in carrying out these assays.

### CROSS REFERENCE TO RELATED APPLICATIONS

The present application claims priority to, and the benefit of, U.S. provisional application 60/929,775, filed on Jul. 12, 2007, the contents of which is hereby incorporated by reference in its entirety.

### FIELD OF THE INVENTION

The invention is in the field of compositions and methods that can be used in diagnosing, and assessing the progression of, inflammatory myopathies.

### BACKGROUND OF THE INVENTION

Inflammatory myopathies are a group of diseases that involve inflammation of muscles or associated tissues and which are characterized primarily by weakness, muscle atrophy and, sometimes, pain. The three major subtypes of inflammatory myopathy are dermatomyositis (DM), polymyositis (PM), and inclusion body myositis (IBM). PM and DM are clinically similar except that DM is associated with skin rashes whereas PM is not. Although DM has been characterized as a disease caused by antibody attack of endothelial antigens (Dalakas, et al., Lancet 362(9388):971-82 (2003)), no well characterized pathogenic antibodies or endothelial antigens have been identified (Greenberg, et al., Curr. Opin. Neurol. 17(3):359-64 (2004)).

IBM is often mistakenly diagnosed as PM but, unlike PM or DM, typically does not respond to treatment with immunosuppressive drugs. Diagnosis of IBM is usually based upon biopsy results revealing muscle cells with inclusion bodies, i.e. with vacuoles containing amyloid protein. There currently is no effective treatment for IBM but patients may sometimes benefit from the administration of prednisone or intravenous immunoglobulin (IVIG).

Gene expression profiling of muscle in adult DM compared with other inflammatory myopathies and normal healthy controls has revealed a gene transcriptional signature that is dominated by the upregulation of interferon-α/β (IFN-α/β)-inducible genes Greenberg, et al., Ann. Neurol. 57(5):664-78 (2005)). Plasmacytoid dendritic cells (PDCs), natural IFN-α/βproducing cells, are present in DM muscle. The interferon-α/β induced protein, MxA, is expressed in perifascicular myofibers and capillaries. These observations suggest that tissue damage in DM derives from a self-destructive over-activation of the innate immune system (Greenberg, et al., Ann. Neurol. 57(5):664-78 (2005)). Further characterization of gene expression patterns in DM, PM and IBM may lead to better methods for distinguishing the inflammatory myopathies from one another and from other diseases characterized by progressive muscle weakness.

### SUMMARY OF THE INVENTION

The present invention is based upon the quantitative measurement of blood RNA transcripts produced, inter alia, by each of the genes IFI27, IFI44L, EPSTI1, CMPK2/LOC129607, IFI44, HERC5, ISG15, MX1, SAMD9L, OAS1, OAS2, OAS3, OASL, IFIT1, IFIT2, IFIT3, IFIT5, BIRC4BP, PLSCR1, TNFAIP6, TNFSF10, CHMP5, IFIH1, EIF2AK2, and GBP1. A high level of any of these transcripts has high positive predictive value for a diagnosis of dermatomyositis or polymyositis, and excludes a diagnosis of the related inflammatory myopathy inclusion body myositis (IBM). High levels of transcripts for any of these genes are not seen in other muscle conditions, such as muscular dystrophy or myasthenia gravis, that may be important to distinguish diagnostically. Of the gene transcripts identified, IFI27 has the strongest predictive value and accuracy. This gene has been previously reported as overactive in DM muscle (Greenberg, et al., Ann. Neurol. 57(5):664-78 (2005)). Three other genes—RSAD2, IFI44L, and EPSTI1 are also excellent choices for a single gene based test.

In its first aspect, the invention is directed to a method for determining whether a subject exhibits a gene expression pattern characteristic of polymyositis, dermatomyositis, or inclusion body myositis by obtaining a test biological sample of peripheral blood mononuclear cells or, less preferably, muscle tissue (e.g., from a biosy) and assaying the sample for the expression of one or more of the following genes (named based upon the product they produce): interferon alpha-inducible protein 27; interferon-induced protein 44-like; radical S-adenosyl domain/CIG5; interferon-induced protein 44; CMPK2 (hypothetical protein LOC129607); 2′,5′-oligoadenylate synthetase 1; epithelial stromal interaction 1; XIAP associated factor-1; interferon-induced tetratricopeptide 1, interferon-induced tetratricopeptide 2, interferon-induced tetratricopeptide 3, interferon-induced tetratricopeptide 5; 2′-5′-oligoadenylate synthetase-like; 2′-5′-oligoadenylate synthetase 3; phospholipid scramblase 1; hect domain and RLD 5; interferon-inducible protein kinase EIF2AK2; TNF superfamily, member 10 (TNFSF10); guanylate binding protein 1; TNF, alpha-induced protein 6; sterile alpha motif domain 9-like; chromatin modifying protein 5; ISG15 ubiquitin-like modifier; T-5′-oligoadenylate synthetase 2; interferon induced helicase C domain 1; and myxovirus resistance 1. Names of the genes, abbreviations for each and accession numbers for sequences in UniGene and GenBank databases are provided in Table 4.

For the purposes of the present invention, assays for determining the level of expression of a gene may be directed either at nucleic acids (e.g., using PCR amplification of mRNA) or at gene products (e.g., using an ELISA or radioimmunoassay). The results obtained using the test sample are compared with results from one or more control samples selected using criteria well known in the art. The control samples may be, for example, samples of blood, serum or plasma derived from individuals known to be free of an inflammatory myopathy or other muscle disease or they may be taken from the population as a whole and, optionally, matched with the test sample with respect to the age of the subject, sex, etc. By comparing the results from the controls and the test sample, a conclusion can be drawn with regard to whether the subject has an inflammatory myopathy and, if present, the particular type of inflammatory myopathy. The more genes exhibiting differences and the greater the magnitude of the differences, the greater the risk of a subject being positive for disease. Preferably assays will be performed on at least 3 genes and, more preferably, at least 5, 10 or 15 genes. An increase of at least 2 fold in samples derived from subjects, compared to controls, should generally be observed in patients with an inflammatory myopathy with greater increases (4, 6, 10 or 20 or 50 fold) being more characteristic of DM or PM. The most preferred genes listed in Table 2 are those exhibiting the greatest difference in expression level in disease carrying individuals relative to controls.

The method described above may also be used in other ways. For example, assays can be performed on patients already diagnosed as having polymyositis, dermatomyositis, or inclusion body myositis for the purpose of tracking disease progression or the effect of a therapeutic regime. In addition, the method can be used to help distinguish DM (in which increases in expression levels are most pronounced); PM (in which increases in gene expression levels are much more modest) and IBM (in which there is still elevated gene expression but to a much lesser degree than the increase seen in either DM or PM).

In another aspect, the invention is directed to a microarray plate or slide having a series of distinct, immobilized oligonucleotides recognizing the sequences of the genes listed above. The term “distinct” indicates that the oligonucleotides have different sequences that allow them to hybridize to different complementary sequences. Many methods are known in the art for producing plates or slides of this nature and any of these methods are compatible with the present invention. The plates or slides must include immobilized oligonucleotides that hybridize under stringent conditions to at least one of the genes set forth above, and, preferably, slides include several distinct oligonucleotides binding to different genes. The term “stringent conditions” indicates conditions that essentially only permit hybridization to occur with the exact complementary sequence of the immobilized oligonucleotide. In general, these hybridizations are performed in buffers of about neutral pH containing 0.1-0.5 NaCl and at a temperature of between 45 and 70° C. It is also possible to carry out incubations under conditions of low stringency and then to use high stringency wash conditions to cause the dissociation of hybridized sequences that are not exact matches. Procedures for carrying out incubations of this type in connection with microarray plates or slides are well known in the art.

Each group of immobilized oligonucleotides hybridizing to a specific gene will occupy a separate location on the microarray plate or slide and in total, there should be no more than 500 distinct oligonucleotides present. In preferred embodiments, there are at least 10 distinct oligonucleotides immobilized on plates that hybridize under stringent conditions to different genes with 15 or 20 such immobilized oligonucleotides being preferred. For economic reasons, it is also preferred that the total number of immobilized sequences present be 500 or less, more preferably 100 or less and more preferably, 50 or less.

The microarray plates described above may be used in carrying out any of the methods of analyzing samples discussed herein. One way of carrying out an analysis would involve lysing cells and then amplifying the mRNA released in the presence of a detectable label, e.g., a nucleotide bound to a dye or other marker and present in a PCR primer. Thus, a population of labeled cDNAs is obtained that can be used directly in hybridizations with oligonucleotides immobilized on a microarray plate or slide. It is also possible to compare two different populations of mRNAs by carrying out PCR in the presence of different dyes for each population. After hybridizations are completed, plates are analyzed using an automated reader to determine the amount of label associated with each immobilized sequence, which reflects the abundance of the hybridized sequence in the original lysate.

Many variations of this basic procedure have been described in the art and are compatible with the present invention.

### DETAILED DESCRIPTION OF THE INVENTION

### I. Preparation of Samples for Use in Hybridizations

Samples containing peripheral blood mononuclear cells (PBMCs) may be obtained, lysed and extracted to obtain RNA using procedures described herein and that are standard in the art. Assays may be performed using standard procedures described by microarray plate manufactures and, in general, as described previously Greenberg, et al., Neurology 59(8):1170-82 (2002)). Alternatively, mRNA can be obtained from muscle cells obtained during biopsies and analyzed.

### II. Microarray Materials and Assays

All of the genes identified herein as being altered in polymyositis or dermatomyositis patients were present on the Affymetrix plates described in the Examples section. In principle, the same plates could be used for evaluating the mRNA from PBMCs or muscle cells. However, since the plates used include immobilized oligonucleotides for thousands of different genes, the system is inconvenient and unnecessarily expensive. Plates better suited to the analysis discussed herein can be made by focusing on the genes listed in Table 2. Thus, plates similar to the Affymetrix plates may be used, under the same assay conditions, but with only a small number of distinct hybridization sites (e.g., 5-50 or 5-100). This simplifies the analysis and allows for replicates to be included to better check on the consistency of results.

Although the same procedures and hardware described by Affymetrix could be employed in connection with the present invention, other alternatives are also available. Many reviews have been written detailing methods for making microarrays and for carrying out assays (see, e.g., Bowtell, Nature Genetics Suppl. 21:25-32 (1999); Constantine, et al., Life Sci. News 1:11-13 (1998); Ramsay, Nature Biotechnol. 16:40-44 (1998)). In addition, patents have issued describing techniques for producing microarray plates, slides and related instruments (U.S. Pat. No. 6,902,702; U.S. Pat. No. 6,594,432; U.S. Pat. No. 5,622,826) and for carrying out assays (U.S. Pat. No. 6,902,900; U.S. Pat. No. 6,759,197). The two main techniques for making plates or slides involve either polylithographic methods (see U.S. Pat. No. 5,445,934; U.S. Pat. No. 5,744,305) or robotic spotting methods (U.S. Pat. No. 5,807,522). Other procedures may involve inkjet printing or capillary spotting (see, e.g., WO 98/29736 or WO 00/01859).

The substrate used for microarray plates or slides can be any material capable of binding to and immobilizing oligonucleotides including plastic, metals such a platinum and glass. A preferred substrate is glass coated with a material that promotes oligonucleotide binding such as polylysine (see Chena, et al., Science 270:467-470 (1995)). Many schemes for covalently attaching oligonucleotides have been described and are suitable for use in connection with the present invention (see, e.g., U.S. Pat. No. 6,594,432). The immobilized oligonucleotides should be, at a minimum, 20 bases in length and should have a sequence exactly corresponding to a segment in the gene targeted for hybridization.

### III. Comments Regarding Additional Methodology

Although the methods described above may be used to determine the level of gene expression in PBMCs and muscle cells, any other procedure for conducting this analysis may also be used in connection with the invention. For example, DNA blotting techniques, with or without PCR amplification, may be used to quantitate levels of genes. Western blots or immunoassays may be used to quantitate gene products and, in some cases, enzyme-based assays may be used. The level of expression can also be assessed by immunofluorescence techniques or promoter based reporter assays. The essential element of the procedure is not how quantitation is performed, but rather the particular genes being examined and the determination of whether those genes are being expressed at a level characteristic of the presence or state of PM, DM or IBM.

### V. Comments Regarding Utility

The assays described herein are designed to assess whether PBMCs or muscle cells have a gene expression profile indicative of the presence or progression of polymyositis, dermatomyositis or inclusion body myositis. This is clearly of great value to scientists that are studying these diseases, and to clinicians trying to make a diagnosis or to determine whether a treatment regimen or drug is having a beneficial effect.

### EXAMPLES

Affymetrix whole genome microarrays were used to measure the expression of approximately 38,500 genes in 65 blood and 15 muscle samples from 56 subjects with dermatomyositis, polymyositis, inclusion body myositis, myasthenia gravis, muscular dystrophy and healthy volunteers. In addition, nine paired blood samples from the same individuals at different times with differing disease activity were compared. Bioinformatics techniques were used to identify genes with significant differential gene expression among diagnostic categories and in relationship to disease activity. The microarray data was corroborated with quantitative real-time PCR.

Most patients with active dermatomyositis and polymyositis, but not patients with inclusion body myositis, were found to have significant and high upregulation of the type 1 interferon-α/β-inducible genes in blood. Furthermore, the upregulation of these genes correlates with disease activity in dermatomyositis and polymyositis, with downregulation occurring when disease is controlled with treatment.

Materials and Methods:

Study Subjects

65 microarray experiments were performed on blood samples from a total of 56 prospectively enrolled subjects of which 36 had inflammatory myopathies (12 with DM, 11 with PM, and 13 with IBM). For additional control groups, we studied five patients with myasthenia gravis, a non-inflammatory autoimmune myopathy, three patients with genetically determined myopathies (two with myotonic dystrophy type 2 and one with mitochondrial myopathy), and 12 healthy subject volunteers. Six patients with DM and two with PM provided second blood samples for microarray experiments performed at two different time points, one where there was active disease, the other when disease was improving; one patient with refractory DM provided two samples at different time points both when there was active disease. All patients met research criteria for definite or probable DM or PM (Hoogendijk, et al., Neuromuscul. Disord. 14(5):337-45 (2004)) and definite or possible IBM (Griggs, et al., Ann. Neurol. 38(5):705-13 (1995)). Patients with systemic lupus erythematosus were excluded. The clinical features of the patients with DM (mean age 47 years) and PM (mean age 54 years) are outlined in Table 1. Six patients, two with DM and four with PM, had interstitial lung disease. Of these, the two DM and one of the PM patients additionally had anti-histidyl transfer RNA (anti-Jo-1) antibodies. Patients with IBM, five men and seven women, had an average age of 69 years, and none were receiving immunomodulatory medication. Healthy volunteers, at the time of recruitment, had not had any serious illness in the last six months, had not started any new medications in the last six months and had no serious cold, flu or other infections in the previous two months. The volunteers were made up of five men and seven women and had an average age of 46 (range 30-62). An Internal Review Board approved the study. Written informed consent was obtained from all participating patients and healthy volunteers.

Assessment of Disease Activity

We classified the DM and PM patients as those with active disease (DMA; PMA) and those with improving disease (DMI; PMI). Those patients who had 3 of the following 4 were classified as active: (1) increasing symptoms, (2) increasing objective weakness on manual muscle testing, (3) elevated and, if more than one measurement available, increasing serum creatine kinase (CK) level and (4) the treating physician increased the patient's immunotherapy. Similar features have been previously used to define active disease in myositis (Nagy, et al., Immunol. Lett. 74(3):207-10 (2000)). DM and PM patients were classified as active or improving prospectively, prior to analysis of gene expression data. Manual muscle testing using MRC grading was used to assess strength; a composite score for 30 different muscle groups was calculated, giving a maximum score of 150. We used the Myositis Intention to Treat Activity Index (MITAX) (Isenberg, et al., Rheumatology (Oxford) 43(1):49-54 (2004)), as proposed by the International Myositis Assessment and Clinical Studies group as an additional measure of disease activity. The MITAX is a multi-system assessment tool looking at the muscle, mucocutaneous, gastrointestinal, respiratory and musculoskeletal systems. Good inter-rater reliability has been reported for this measure of disease activity. In the nine patients with paired samples we saw an average reduction in MITAX score of 8.5 between active and improving. Active scores ranged from 12-13 and improving scores ranged from 2-6.

PBMC Collection, Muscle Tissue Collection, and RNA Extraction

We collected 10 mls of blood from patients and volunteers into EDTA-containing tubes (57 samples), or in some cases directly into PAX-Gene-RNA tubes (8 samples). For the EDTA-containing tubes, after centrifugation, we aspirated the plasma (upper layer) down to 1 mm from the red blood cells, and then carefully aspirated 500 μl of buffy coat into Cryostat Storage Tubes, already filled with 1.2 mls of solution of RNAlater (Ambion, Austin, Tex.). We froze the combined buffy coat and RNAlater at −20° C. RNA was extracted using “Ribo Pure” (Ambion, Austin, Tex.) from the buffy coat and from PAX-Gene RNA tubes. RNA concentration was measured using a spectrophotometer, and RNA quality was evaluated by running 1 μg of RNA on 1% agarose gels. Muscle biopsy samples weighing 70 to 120 mg had RNA extracted as previously described (Greenberg, et al., Neurology 59(8):1170-82 (2002)). Muscle biopsy tissue was obtained from 15 patients (5 DM, 5 PM, and 5 IBM) at the time of active disease, all of whom also had blood microarray studies at active or improving time points, and from 5 patients without neuromuscular disease undergoing diagnostic biopsies. Muscle RNA extraction was done with RiboPure similarly to PBMC RNA extraction. Of these 15 inflammatory myopathy muscle microarray studies, 9 (3 with DM, 2 with PM, and 4 with IBM) were previously performed with portions of the data used in publication, and reanalyzed in this study, and 6 were newly performed specifically for these studies.

Target Preparation, Hybridization, and Signal Detection

Microarray studies were performed for muscle as previously described, using Affymetrix HG-U133A microarrays (Greenberg, et al., Neurology 59(8):1170-82 (2002)). PBMC samples were processed using Affymetrix HG-U133A plus 2.0 microarrays and GeneChip Operating System (GCOSv1.3) version 1.3.

Data Processing

The Affymetrix HG-U133 plus 2.0 GeneChip has 54,675 probe sets including 63 control probe sets. Probe set annotations were obtained from NetAffx Analysis Center, version Mar. 9, 2007. The expression levels were calculated using GC-Content Robust Multichip Analysis (GCRMA), which was implemented in the Bioconductor GCRMA package (available at http://www.bioconductor.org/download/oldrelease/bioc1.6/popular/gcrma.html). This algorithm produces an improved expression measurement by accounting for GC-content based bias and optical noise behavior from all the arrays in an experiment (Wu, et al., J. Comput. Biol. 12(6):882-93 (2005)). Quality control was performed by visual inspection of scanned and reconstructed images to identify gross artifact and by careful assessment of the quality assessment parameters including control probe sets. All blood and muscle microarray data were analyzed together with GCRMA in this study.

Data Analysis and Visualization

The average and 90% confidence intervals (CIs) of fold changes were calculated for each disease group compared to control group in addition to p-value of two group comparisons using Welch's t-test (Table 2). We applied stringent criteria to select genes as significantly upregulated, requiring a p-value<0.0001, and the lower bound of CIs>4.0. Genes were identified as IFN-α/13 induced through searches of literature (10-12) and molecular databases.

Group fold changes and CIs were calculated comparing 8 DMA, 11 DMI, 7 PMA, 6 PMI, 13 IBM, 5 MG, 3 genetically determined myopathies, and 12 normal blood specimens. Additionally, 9 patients (7 with DM, 2 with PM) with paired samples (18 samples) were analyzed pair-wise for treatment associated changes in gene signatures. Blood and muscle expression data were compared for 13,398 genes common to both HG-U133A and HG-U133A plus 2.0 microarray chips mapped according to Affymetrix probeset identifications.

Quantitative Real-Time Polymerase Chain Reaction (qRT-PCR)

We performed quantitative real-time PCR for two IFN-inducible genes: IFIT1 and MX1 on 18 samples (4 DMA, 5 DMI, 4 IBM, and 5 healthy volunteers) using primers designed with Primer software (Whitehead Institute, Cambridge, Mass.) and purchased commercially (Operon Biotechnologies, Inc. Huntsville, Ala.). Primers used were as follows: MxA: forward 5′-CGGCTAACGGATAAGCAGAG-3′ (SEQ ID NO:1), and reverse 5′-ACCTACAGCTGGCTCCTGAA-3′ (SEQ ID NO:2; IFIT1: forward 5′-AAAAGCCCAC-ATTTGAGGTG-3′ (SEQ ID NO:3), and reverse 5′-GAAATTCCTGAAACCGACCA-3′ (SEQ ID NO:4).

RNA (1 μg) was reverse-transcribed to cDNA with oligo(dT)20 and Ready-to-Go reverse transcription kit from (Amersham Biosciences, Piscataway, N.J.). SYBR Green I-based real-time PCR was carried out on Opticon Monitor (MJ Research, Inc, Waltham, Mass.) with cDNA templates (1/100 of the RT reaction) using Promega (Madison, Wis.) taq polymerase and buffer, 2 mM MgCl2, 400 mM deoxy-NTP (Roche), 0.5×SYBR Green I, 0.8 mM of each PCR primer (Operon), in a 25 ml final volume reaction. The samples were loaded into wells of Low Profile 96-well microplates. After an initial denaturation step at 95° C. for 5 min, conditions for cycling were 40 cycles of denaturation (95° C. for 30 s), annealing (for 30 s), and extension (72° C. for 1 min). The fluorescence signal was measured immediately after incubation at 79° C. for 5 s following each extension step, eliminating possible primer dimer detection. At the end of PCR cycles, a melting curve was generated to confirm the specificity of the PCR product. For each run, serial dilutions of human GAPDH plasmids were used as standards for quantitative measurement of the amount of amplified cDNA. All PCR reactions were run in triplicate. Comparative CT method was used to quantify the amplified transcripts. Mean fold ratios of amplified transcripts were calculated comparing DMI/DMA, DMA/Normal, DMA/Normal and IBM/Normal.

Immunohistochemistry

Frozen muscle sections from 15 patients (5 each with DM, PM, and IBM) whose muscle underwent microarray studies were stained with antibodies against myxovirus resistance A (anti-MxA antibodies; courtesy of Dr. Otto Haller, Department of Virology, University of Freiburg, Germany) as previously described (Greenberg, et al., Ann. Neurol. 57(5):664-78 (2005)) and examined for correlation with transcript studies.

Results

Blood Interferon-α/β-Inducible Gene Transcripts are the Most Upregulated of all Genes in PBMCs from Patients with Active DM and to a Lesser Extent Active PM

Comparing transcript expression levels for active DM (DMA) and PM (PMA) with healthy controls, genes induced by interferon-α/β had the largest fold changes and highest statistical significance among the approximately 38,500 measured transcripts (p values<0.0001) (Table 2). Of the 25 most highly upregulated genes, at least 21 (84%) are known to be interferon-α/β-inducible. None of these genes were significantly upregulated in patients with IBM, MG or genetically determined myopathies. The magnitude of upregulation was generally higher in DM than in PM. Quantitative RT-PCR showed that the interferon-inducible genes Mx1 and IFIT1 were highly upregulated in DMA blood supporting our observations from the microarray data (Table 3). The average coefficient of variance of triplicate samples was 0.15, with a high correlation between triplicate runs of 0.99. Correlation of the RT-PCR data with microarray data was excellent (Mx1: R²=0.9889; IFIT1: R²=0.9978). Overall, 8 of 8 patients with DMA and 5 of 7 patients with PMA had levels of overexpression of interferon-α/β-inducible genes that exceeded that of all other 50 blood specimens studied.

Interferon-α/β-Inducible Genes are Downregulated with Clinical Improvement in DM and PM

We compared transcript profiles of 8 DMA samples with those of 11 DMI, and separately 7 PMA samples with 6 PMI. The genes most highly downregulated with improvement in disease are predominantly interferon-α/β-inducible (Table 2, right columns). Quantitative RT-PCR similarly confirmed improvement in DM patients (Table 3). In paired samples from the same patients with DM (N=6) or PM (N=2) who had active and improving disease at two different time points, the type 1 interferon-inducible genes were again the most downregulated of all genes. For the one patient with refractory DM, little overall change for many type 1 interferon-inducible genes was observed in the paired specimens.

Upregulation of Interferon-α/β-Inducible Genes is Greater in Muscle than in Blood in DM but not in PM or IBM

For 15 patients (5 each with DM, PM, and IBM), we compared the blood gene expression profiles with muscle gene expression using the 13,398 genes which are shared among both the U133A (used for muscle profiling) and U133 plus 2.0 (used for blood profiling) microarrays. In DM muscle, there is marked upregulation of the expression of the same interferon-α/β-inducible genes that we found to be highly upregulated in blood. In contrast, in PM and IBM muscle only a modest increase of the interferon-α/β-inducible gene transcription was present. This may have been due to infiltrating immune system cells that themselves express interferon-α/β-inducible genes, such as MxA. Of particular interest in DM, there is a marked overexpression of certain interferon-inducible genes in muscle in comparison to blood. For example, DM muscle expression of ISG15 was approximately 570 times that of normal muscle and about 100 fold higher than in DM blood.

Upregulation of Interferon-60-Inducible MxA Protein Correlates with Tissue Pathology

As previously reported, the overexpression of one interferon-α/β-inducible gene protein, MxA, is present in muscle in DM (Greenberg, et al., Ann. Neurol. 57(5):664-78 (2005)). In the current study, MxA transcript level, although similarly elevated in DMA-blood (6.2 fold) and PMA-blood (6.0 fold), was markedly higher by microarray studies in DM-muscle (281 fold) compared to PM-muscle (2.5 fold). The marked enrichment of MxA transcript in DM muscle is similarly accompanied by marked enrichment of MxA protein by immunohistochemistry in comparing muscle sections from DM and PM. In 4 of 5 DM patients, MxA staining was present intensely in many myofibers, particularly perifascicular myofibers, while in all 5 patients with PM and 5 with IBM, MxA staining was limited to infiltrating immune system cells. MxA staining is not present in normal muscle biopsies.

Discussion

Our findings suggest that, in most patients with DM and PM, but not in patients with IBM, there is a distinct blood gene expression profile characterized by marked overexpression of interferon-α/β-inducible genes. Clinical improvement during immunosuppressive treatment is generally associated with reduction in the overexpression of these genes towards normal levels. These findings, in relationship to gene expression in muscle, have implications for pathogenic hypotheses and blood biomarkers of potential diagnostic use.

For dermatomyositis, the interferon-α/β gene signature in blood is highly correlated with the findings of microarray studies in muscle and supports the hypothesis that this disease may be driven by systemic and intramuscular overproduction of interferon-α/β. Similar blood gene transcription signatures have been reported in systemic lupus erythematosus (SLE) (Baechler, et al., Proc. Nat'l Acad. Sci. USA 100(5):2610-5 (2003); Bennett, et al., J. Exp. Med. 197(6):711-23 (2003); Han, et al., Genes Immun. 4(3):177-86 (2003)). Overexpression at the protein level for at least one of these genes (MxA) is present in DM muscle capillaries and perifascicular myofibers, and DM skin (Wenzel, et al., Br. J. Dermatol. 153(2):462-3 and 463-4 (2005); Wenzel, et al., Clin. Exp. Dermatol. 31(4):576-82 (2006)). Additionally plasmacytoid dendritic cells (pDCs), natural IFN-α producing cells, are abundant in DM muscle (Greenberg, et al., Ann. Neurol. 57(5):664-78 (2005)) and skin (Wenzel, et al., Clin. Exp. Dermatol. 31(4):576-82 (2006)). Upregulation of MxA transcript levels in blood have been observed in juvenile DM and may correlate with disease activity (O'Connor, et al., Clin. Immunol. 120(3):319-25 (2006)).

Although blood profiles exhibited similar levels of overexpression of interferon-α/β-inducible genes in both DM and PM, in muscle some of these genes are orders of magnitude more highly expressed only in DM. One explanation for this could be that although systemic activation of the innate immune system is present in both diseases, DM muscle is exposed to a greater amount of type 1 interferons than PM muscle. This hypothesis is supported by previous findings of interferon-α/β secreting plasmacytoid dendritic cells infiltrating DM muscle (Greenberg, et al., Ann. Neurol. 57(5):664-78 (2005)) in much greater numbers than seen in IBM and PM (Greenberg, et al., Muscle Nerve 35(1):17-23 (2007)). Additionally, whereas in PM the expression of the interferon-α/β-inducible protein MxA is confined to invading inflammatory cells, in DM MxA protein is present within myofibers.

The enrichment of such specific interferon-α/β-inducible genes in muscle likely is an important clue to the nature of tissue injury in DM. Thus, the marked enrichment of ISG15 transcript in DM muscle suggests that of the various interferon-α/β-inducible proteins upregulated in DM blood and muscle, this particular molecule, a ubiquitin-like modifier, could be of greater relevance to the direct mechanisms of tissue injury in DM.

The distinct lack of highly upregulated interferon-α/β genes in IBM blood, compared with PM blood, contrasts with the otherwise similar nature of immunological abnormalities that have previously been observed in muscle in these two diseases. These findings suggest a different magnitude of activation of the innate immune system in PM than IBM. Further study of this hypothesis would best be addressed in larger numbers of patients. Additionally, for many patients the diagnosis of IBM is delayed, recognized only after a previous diagnosis of glucocorticoid-resistant PM (Amato, et al., Ann. Neurol. 40(4):581-6 (1996)). Further characterization of the interferon-α/β-inducible gene blood biomarkers in IBM and PM suggests the potential for future earlier diagnosis of IBM and avoidance of glucocorticoid treatment for such patients.

Our findings also suggest the utility of blood biomarkers of disease activity to supplement management of patients with DM and PM. In this study, we have identified multiple blood biomarkers of active medication responsive myositis. Currently there is a need for more specific tests to evaluate disease activity in DM or PM. The level of serum creatine kinase (CK) is generally reflective of disease activity in PM, but may be normal in patients with active DM. The MITAX has been proposed as a clinical measure of disease activity. We calculated a MITAX score for our DM patients, which correlated well with our own assessment of disease activity. However, while the MITAX has been shown to be a good tool for disease activity assessment, intra-class correlation between assessors for muscle involvement was low, underlining the need for a more objective measure (Isenberg, et al., Rheumatology (Oxford) 43(1):49-54 (2004)). An objective and inexpensive PCR based blood test which correlates the expression of certain interferon-α/β-inducible genes with disease activity could supplement the clinical management of DM and PM. Eventually such tests might provide surrogate markers for treatment response in clinical trials.

TABLE 1
Clinical features of 23 patients with dermatomyositis and polymyositis.

| | | | | | | | | | | | | |
|---|---|---|---|---|---|---|---|---|---|---|---|---|
| Patient ID/ | | | | Treatment | | | | MMT | | | | Other |
| Disease | A/S | PA | DD | (Tx) | Tx-D | CK | S | score | X | Y | Z | Diagnoses |

Dermatomyositis (DM) N = 12

| | | | | | | | | | | | | |
|---|---|---|---|---|---|---|---|---|---|---|---|---|
| BGE10-DMA* | 38/F | No | 108 | Pred/IVIG | 4 | 948 | ↑ | 142 | ↓ | ↑ | 12 | — |
| BGE19-DMA* | 61/F | Yes | 12 | Pred/Myco | 24 | 165 | ↑ | 139 | ↓ | ↑ | 13 | Breast ca |
| BGE46-DMA* | 25/F | Yes | 8 | Pred | 0.1 | 393 | ↑ | 128 | ↓ | ↑ | 12 | — |
| BGE79-DMA* | 46/F | No | 72 | None | 0 | 740 | ↑ | 133 | ↓ | ↑ | 12 | Calcinosis |
| BGE92-DMA* | 27/F | Yes | 17 | Pred | 11 | 1140 | ↑ | 146 | ↓ | ↑ | 13 | ILD, Jo-1 |
| BGE95-DMA* | 53/M | Yes | 25 | MTX | 23 | 6416 | ↑ | 131 | ↓ | ↑ | 13 | Diabetes |
| BGE99-DMA* | 21/M | Yes | 3 | None | 0 | 352 | ↑ | 148 | ↓ | ↑ | 12 | — |
| BGE110-DMA | 54/F | Yes | 6 | Pred | 2 | 1140 | ↑ | 146 | ↓ | ↑ | 13 | ILD, Jo-1 |
| BGE15-DMI | 62/F | Yes | 36 | Pred/Myco | 12 | 80 | ↓ | 150 | f | ↓ | 2 | |
| BGE17-DMI | 70/F | No | 24 | Pred/IVIG/ | 6 | 19 | ↓ | 123 | ↑ | ↓ | 2 | — |
| | | | | Myco | | | | | | | | |
| BGE36-DMI | 59/F | No | 12 | Pred/Myco | 1 | 1652 | ↓ | 140 | ↓ | ↓ | 2 | — |
| BGE80-DMI | 44/F | No | 1 | Pred | 1 | 71 | ↓ | 140 | ↓ | ↓ | 2 | — |

Polymyositis (PM) N = 11

| | | | | | | | | | | | | |
|---|---|---|---|---|---|---|---|---|---|---|---|---|
| BGE3-PMA | 55/F | No | 26 | Pred/Myco | 20 | 2100 | ↑ | 140 | ↓ | ↑ | na | — |
| BGE32-PMA | 72/F | No | 3 | Pred/IVIG | 2 | 1219 | ↑ | 100 | ↓ | ↑ | na | — |
| BGE47-PMA | 72/F | No | 11 | Pred/Myco | 9 | 3027 | ↑ | 118 | ↓ | ↑ | na | — |
| BGE98-PMA | 38/F | No | 16 | Pred | 16 | 129 | ↑ | 139.66 | ↓ | ↑ | na | MCTD |
| BGE106-PMA* | 59/F | No | 2 | None | 0 | 4720 | ↑ | 128.65 | ↓ | ↑ | na | — |
| BGE119-PMA* | 47/F | No | 5 | None | 0 | 1256 | ↑ | 135 | ↓ | ↑ | na | ILD/MCTD |
| BGE121-PMA | 67/M | No | 12 | None | 0 | 1102 | ↑ | 137 | ↓ | ↑ | na | ILD |
| BGE11-PMI | 65/F | No | 81 | Aza/IVIG | 48 | 472 | ↓ | 149 | ↑ | ↓ | na | ILD, Jo-1 |
| BGE26-PMI | 71/F | No | 29 | Pred/Aza/ | 29 | 29 | ↓ | 149 | ↑ | ↓ | na | ILD |
| | | | | IVIG | | | | | | | | |
| BGE50-PMI | 40/F | No | 2 | Pred/IVIG | 2 | 663 | ↓ | 138 | ↑ | ↓ | na | — |
| BGE58-PMI | 30/M | No | 25 | Pred/Mtx/ | 25 | 1743 | ↓ | 119 | ↑ | ↓ | na | — |
| | | | | IVIG | | | | | | | | |

Paired blood specimens were studied from those 9 patients marked with an asterisk (*) for a total of 32 blood samples from patients with DM and PM.
A/S = Age/sex; AZA = azathioprine; CK = creatine kinase; DD = Disease duration; DMA = DM active; DMI = DM improving; DMS = DM sine myositis; f = full; IVIG = intravenous immunoglobulin; MCTD = mixed connective tissue disease; ILD = interstitial lung disease; Meds = medications; MMT = manual muscle testing; PA = Perifascicular atrophy; Δ = change; PMA = PM active; PMI = PM improving; Pred = prednisone; Myco = mycophenolate mofetil; MTX = methotrexate; S = symptoms; MITAX = Myositis Intention to Treat Activity Index; Tx-D = Disease duration (months); X = Δ MMTscore; Y = Δ Meds; Z = MITAX score (DM only)

TABLE 2
Interferon-α/β-inducible genes are the most highly overexpressed genes
in active dermatomyositis and polymyositis, but not other control groups.

| | | | | | | | | Improving/Active | |
|---|---|---|---|---|---|---|---|---|---|
| Gene | | Disease Group Comparisons | | | | | | Comparison | |
| Symbol | Gene Title | A | B | C | D | E | F | G | H |
| IFI27 | interferon alpha-inducible prt 27 | 7.94E−06 | 130 (28-277) | 21.65 | 2.79 | 1.01 | 0.94 | −2.34 | 1.31 |
| IFI44L | interferon-induced prt 44-like | 2.84E−07 | 104 (39-194) | 64.23 | 7.01 | 0.68 | 4.09 | −9.65 | −5.16 |
| RSAD2 | radical S-adenosyl | 2.20E−07 | 66 (26-154) | 42.04 | 4.63 | 1.09 | 2.98 | −9.76 | −6.00 |
| | domain/CIG5 | | | | | | | | |
| IFI44 | interferon-induced prt 44 | 1.61E−06 | 57 (17-104) | 27.28 | 2.94 | 0.52 | 1.46 | −21.26 | −7.11 |
| LOC129607 | hypothetical prt LOC129607 | 4.74E−09 | 47 (20-114) | 29.34 | 3.48 | 1.20 | 2.51 | −16.42 | −5.68 |
| OAS1 | 2′,5′-oligoadenylate synthetase 1 | 3.80E−09 | 36 (13-84) | 15.13 | 2.05 | 0.35 | 1.04 | −18.22 | −3.90 |
| EPSTI1 | epithelial stromal interaction 1 | 4.27E−07 | 33 (15-57) | 16.24 | 3.09 | 0.73 | 3.50 | −17.93 | −2.21 |
| BIRC4BP | XIAP associated factor-1 | 7.99E−06 | 24 (12-39) | 17.38 | 4.18 | 0.78 | 1.76 | −13.25 | −3.45 |
| IFIT5 | interferon-induced | 6.81E−06 | 20 (10-32) | 14.65 | 2.63 | 0.89 | 1.98 | −13.24 | −3.65 |
| | tetratricopeptide 5 | | | | | | | | |
| OASL | 2′,5′-oligoadenylate synthetase- | 3.23E−08 | 19 (9-50) | 9.49 | 2.42 | 0.38 | 0.84 | −11.07 | −3.92 |
| | like | | | | | | | | |
| OAS3 | 2′,5′-oligoadenylate synthetase 3 | 7.74E−06 | 19 (8-34) | 12.68 | 1.94 | 0.60 | 1.38 | −13.18 | −5.02 |
| IFIT1 | interferon-induced | 2.87E−06 | 18 (8-47) | 15.50 | 1.92 | 0.37 | 1.45 | −8.93 | −7.83 |
| | tetratricopeptide 1 | | | | | | | | |
| PLSCR1 | phospholipid scramblase 1 | 5.49E−07 | 16 (9-25) | 14.97 | 3.57 | 5.68 | 2.69 | −4.19 | −2.35 |
| HERC5 | hect domain and RLD 5 | 1.21E−06 | 15 (5-30) | 11.62 | 1.91 | 0.49 | 1.13 | −13..58 | −4.14 |
| EIF2AK2 | interferon-inducible prt kinase | 2.78E−06 | 13 (7-24) | 12.18 | 4.59 | 1.52 | 3.16 | −6.23 | −1.76 |
| TNFSF10 | TNF superfamily, member 10 | 6.42E−05 | 13 (7-21) | 10.79 | 3.58 | 2.69 | 3.26 | −5.21 | −2.56 |
| GBP1 | guanylate binding prt 1 | 3.26E−05 | 13 (6-21) | 6.73 | 3.83 | 0.54 | 3.32 | −9.79 | −1.75 |
| TNFAIP6 | TNF, alpha-induced prt 6 | 4.83E−06 | 11 (6-17) | 8.05 | 1.76 | 3.06 | 1.65 | −4.44 | −2.00 |
| IFIT3 | interferon-induced | 5.46E−08 | 9 (6-14) | 7.41 | 1.84 | 0.59 | 1.82 | −5.45 | −2.95 |
| | tetratricopeptide 3 | | | | | | | | |
| SAMD9L | sterile alpha motif domain 9-like | 1.59E−05 | 9 (5-17 | 13.96 | 7.58 | 0.39 | 2.10 | −13.71 | 1.31 |
| CHMP5 | chromatin modifying protein 5 | 4.21E−05 | 9 (5-13) | 6.42 | 1.60 | 1.90 | 1.88 | −4.21 | −2.44 |
| ISG15 | ISG15 ubiquitin-like modifier | 5.24E−05 | 9 (4-15) | 4.86 | 1.04 | 0.19 | 0.55 | −5.05 | −4.62 |
| OAS2 | 2′,5′-oligoadenylate synthetase 2 | 5.94E−05 | 8 (4-14) | 4.15 | 0.94 | 0.38 | 1.23 | −9.74 | −6.46 |
| IFIH1 | interferon induced helicase C | 1.50E−05 | 7 (4-10) | 7.40 | 1.86 | 0.50 | 1.74 | −8.09 | −2.97 |
| | domain 1 | | | | | | | | |
| MX1 | myxovirus resistance 1 | 5.21E−06 | 6 (3-13) | 6.04 | 1.69 | 0.21 | 0.83 | −4.54 | −4.65 |

The highest differentially expressed genes are listed in descending order of fold-ratios for active DM (DMA) compared to normal. The gene symbols of well-established interferon-α/β inducible genes are in bold. The marked upregulation in DMA (N = 8) is also seen in active PM (PMA; N = 7), but not active IBM (N = 13), myasthenia gravis (N = 5), or genetic myopathies (N = 3) compared to normal (Norm; N = 12). Criteria for gene significance was a p-value <0.0001, with the top 25 genes ordered by fold change (with confidence intervals) listed. Comparison of Improving to Active disease groups show downregulation that occurs with treatment related improvement.
prt = protein; A = p-value DMA/norm; B = DMA/norm Fold (confidence interval); C = PMA/norm Fold; D = IBM/norm Fold; E = MG/norm Fold; F = DYS/norm Fold;

TABLE 3
Quantitative Reverse Transcriptase PCR of transcripts Mx1 and IFIT1
Transcripts were in blood from active DM (DMA; N = 4), improving
DM (DMI; N = 5), IBM (N = 4) and healthy volunteers (N = 5);
fold ratios are listed. Transcripts are upregulated in DM compared
to normals and other inflammatory myopathies. Correlation of RT-PCR
with microarray data was excellent (Mx1: R² = 0.9889;
IFIT1: R² = 0.9978).

| Gene Symbol | DMI/DMA | DMA/Norm | DMI/Norm | IBM/Norm |
|---|---|---|---|---|
| IFITI-array | −468 | 15.87 | 1.18 | 0.77 |
| IFITI-rtPCR | −9.31 | 55.55 | 5.97 | 1.77 |
| Mx1-array | −5.47 | 5.93 | 1.97 | 1.29 |
| Mx1-rtPCR | −4.45 | 25.68 | 5.77 | 1.38 |

TABLE 4
List of Blood Gene RNA Transcripts
Genes identified by unique National Center for Bioinformatics UniGene
ID number, which represents all sequences within the GenBank database;
a single GenBank ID number is provided for an example of a
representative sequence.

| Gene Symbol | Gene Name | UniGene ID | GenBank ID |
|---|---|---|---|
| IFI27 | interferon alpha-inducible protein 27 | Hs.532634 | BT006781.1 |
| IFI44 | interferon-induced protein 44 | Hs.82316 | NM_006417.4 |
| IFI44L | interferon-induced protein 44-like | Hs.389724 | NM_006820.2 |
| CMPK2 | Cytidine monophosphae kinase | Hs.7155 | NM_207315.2 |
| EIF2AK2 | Interferon-inducible protein kinase | Hs.131431 | AY302136.1 |
| EPSTI1 | epithelial stromal interaction 1 | Hs.546467 | AF396928.1 |
| BIRC4BP | XIAP associated factor-1 | Hs.441975 | BC058017.1 |
| OAS1 | 2′,5′-oligoadenylate synthetase 1 | Hs.524760 | BT006785.1 |
| OAS2 | 2′-5′-oligoadenylate synthetase 2 | Hs.414332 | BC049215.1 |
| OAS3 | 2′-5′-oligoadenylate synthetase 3 | Hs.528634 | NM_006187.2 |
| OASL | 2′-5′-oligoadenylate synthetase-like | Hs.118633 | AJ225089.1 |
| IFIT1 | interferon-induced tetratricopeptide 1 | Hs.20315 | BT006667.1 |
| IFIT2 | interferon-induced tetratricopeptide 2 | Hs.437609 | AK312831.1 |
| IFIT3 | interferon-induced tetratricopeptide 3 | Hs.714337 | BT007284.1 |
| IFIT5 | interferon-induced tetratricopeptide 5 | Hs.252839 | NM_012420.1 |
| PLSCR1 | phospholipid scramblase 1 | Hs.130759 | AF098642.1 |
| HERC5 | hect domain and RLD 5 | Hs.26663 | AB027289.1 |
| GBP1 | guanylate binding protein 1 | Hs.62661 | BT006847.1 |
| MX1 | myxovirus resistance 1 | Hs.517307 | M30817.1 |
| RSAD2 | radical S-adenosyl domain/CIG5 | Hs.17518 | BC017969.1 |
| SAMD9L | Sterile alpha motif domain 9-like | Hs.489118 | BC127118.1 |
| TNFAIP6 | TNF, alpha-induced protein 6 | Hs.437322 | NM_007115.2 |
| TNFSF10 | TNF superfamily, member 10 | Hs.478275 | NM_003810.2 |
| CHMP5 | chromatin modifying protein 5 | Hs.635313 | AF229832.1 |
| ISG15 | ISG15 ubiquitin-like modifier | Hs.458485 | M13755.1 |
| IFIH1 | interferon induced helicase C domain 1 | Hs.163173 | BC046208.1 |

All references cited herein are fully incorporated by reference in their entirety. Having now fully described the invention, it will be understood by those of skill in the art that the invention may be practiced within a wide and equivalent range of conditions, parameters and the like, without affecting the spirit or scope of the invention or any embodiment thereof.

1. A method of determining whether a subject exhibits a gene expression pattern characteristic of polymyositis or dermatomyositis, comprising:
a) obtaining a test biological sample from said subject containing peripheral blood mononuclear cells;
b) assaying said sample for the expression of one or more genes selected from the group consisting of: interferon alpha-inducible protein 27; interferon-induced protein 44-like; radical S-adenosyl domain/CIG5; interferon-induced protein 44; hypothetical protein LOC129607; 2′,5′-oligoadenylate synthetase 1; epithelial stromal interaction 1; XIAP associated factor-1; interferon-induced tetratricopeptide 5; 2′-5′-oligoadenylate synthetase-like; 2′-5′-oligoadenylate synthetase 3; interferon-induced tetratricopeptide 1; phospholipid scramblase 1; hect domain and RLD 5; interferon-inducible protein kinase; TNF superfamily, member 10; guanylate binding protein 1; TNF, alpha-induced protein 6; interferon-induced tetratricopeptide 3; sterile alpha motif domain 9-like; chromatin modifying protein 5; ISG15 ubiquitin-like modifier; 2′-5′-oligoadenylate synthetase 2; interferon induced helicase C domain 1; and myxovirus resistance 1;
c) concluding that said subject has a gene expression profile characteristic of polymyositis or dermatomyositis if the results determined in the assay of paragraph b) indicate that one or more of said genes are expressed more highly in said test sample than in one or more control samples.

2. The method of claim 1, wherein said test biological sample is a sample of blood, plasma or serum.

3. The method of claim 1, wherein said assay is performed using a microarray plate.

4. The assay of claim 1, wherein the expression of at least 5 of said genes is elevated in said test sample relative to said one or more control samples.

5. (canceled)

6. The assay of claim 1, wherein the expression of at least 15 of said genes is elevated in said test sample relative to said one or more control samples.

7. The assay of claim 1, wherein the expression of all of the genes in paragraph b) is elevated in said test sample relative to said one or more control samples.

8. A method of determining whether a patient with polymyositis or dermatomyositis has a gene expression pattern that is characteristic of disease progression or of disease remission, comprising:
a) obtaining a test biological sample from said patient containing peripheral blood mononuclear cells;
b) assaying said sample for the expression of one or more genes selected from the group consisting of: interferon alpha-inducible protein 27; interferon-induced protein 44-like; radical S-adenosyl domain/CIG5; interferon-induced protein 44; hypothetical protein LOC129607; 2′,5′-oligoadenylate synthetase 1; epithelial stromal interaction 1; XIAP associated factor-1; interferon-induced tetratricopeptide 5; 2′-5′-oligoadenylate synthetase-like; 2′-5′-oligoadenylate synthetase 3; interferon-induced tetratricopeptide 1; phospholipid scramblase 1; hect domain and RLD 5; interferon-inducible protein kinase; TNF superfamily, member 10; guanylate binding protein 1; TNF, alpha-induced protein 6; interferon-induced tetratricopeptide 3; sterile alpha motif domain 9-like; chromatin modifying protein 5; ISG15 ubiquitin-like modifier; 2′-5′-oligoadenylate synthetase 2; interferon induced helicase C domain 1; and myxovirus resistance 1;
c) concluding that said subject has a gene expression profile characteristic of disease progression if the results determined in the assay of paragraph b) indicate that one or more of said genes are expressed more highly in said test sample than in one or more samples obtained from the same patient at an earlier time; or
d) concluding that said subject has a gene expression profile characteristic of disease remission if the results determined in the assay of paragraph b) indicate that one or more of said genes are expressed to a lesser degree in said test sample than in one or more samples obtained from the same patient at an earlier time.

9. The method of claim 8, wherein said biological sample is a sample of blood, plasma or serum.

10. The method of claim 8, wherein said assay is performed using a microarray plate.

11. The assay of claim 8, wherein the expression of at least 5 of said genes must be elevated in said test sample to conclude that said disease is progressing and the expression of at least 5 of said genes must be decreased in said test sample to conclude that said disease is progressing.

12. (canceled)

13. The assay of claim 8, wherein the expression of at least 15 of said genes must be elevated in said test sample to conclude that said disease is progressing and the expression of at least 15 of said genes must be decreased in said test sample to conclude that said disease is progressing.

14. The assay of claim 8, wherein the expression of all of the genes in paragraph b) must be elevated in said test sample to conclude that said disease is progressing and the expression of all of the genes in paragraph b) must be decreased in said test sample to conclude that said disease is progressing.

15. A microarray plate comprising a series of distinct immobilized oligonucleotides, wherein:
a) at least five of said oligonucleotides hybridizes under stringent conditions specifically to a gene sequence selected from the group consisting of: interferon alpha-inducible protein 27; interferon-induced protein 44-like; radical S-adenosyl domain/CIG5; interferon-induced protein 44; hypothetical protein LOC129607; 2′,5′-oligoadenylate synthetase 1; epithelial stromal interaction 1; XIAP associated factor-1; interferon-induced tetratricopeptide 5; 2′-5′-oligoadenylate synthetase-like; 2′-5′-oligoadenylate synthetase 3; interferon-induced tetratricopeptide 1; phospholipid scramblase 1; hect domain and RLD 5; interferon-inducible protein kinase; TNF superfamily, member 10; guanylate binding protein 1; TNF, alpha-induced protein 6; interferon-induced tetratricopeptide 3; sterile alpha motif domain 9-like; chromatin modifying protein 5; ISG15 ubiquitin-like modifier; 2′-5′-oligoadenylate synthetase 2; interferon induced helicase C domain 1; and myxovirus resistance 1;
b) said oligonucleotides that hybridize under stringent conditions specifically to said gene sequence are immobilized at a location on said microarray plate that does not contain any oligonucleotides that hybridize to other sequences under stringent conditions; and
c) said microarray plate comprises no more than 500 distinct immobilized oligonucleotides in total.

16. The microarray plate of claim 15, wherein said plate comprises at least 10 distinct immobilized oligonucleotides that hybridize under stringent conditions specifically to a gene sequence selected from the genes in paragraph b).

17. The microarray plate of claim 15, wherein said plate comprises at least 15 distinct immobilized oligonucleotides that hybridize under stringent conditions specifically to a gene sequence selected from the genes in paragraph b).

18. The microarray plate of claim 15, wherein said plate comprises distinct immobilized oligonucleotides that hybridize under stringent conditions specifically to all of the genes in paragraph b).

19. The microarray plate of claim 18, wherein said microarray plate comprises no more than 100 distinct immobilized oligonucleotides in total.

20. The microarray plate of claim 18, wherein said microarray plate does not comprise any distinct immobilized oligonucleotides that hybridize under stringent conditions to a gene other than a gene in paragraph b).

21. A method of determining the gene expression profile of a subject comprising:
a) obtaining a test biological sample from said subject containing peripheral blood mononuclear cells;
b) assaying gene expression in said test biological sample using the microarray plate of claim 15.

22. A method of diagnosing polymyositis or dermatomyositis in a subject, comprising:
a) obtaining a test biological sample of blood or muscle from said subject;
b) assaying said test biological sample to determine the level of expression of the IFI44L gene and/or EPSTI1;
c) concluding that said subject has polymyositis or dermatomyositis if the level of gene expression determined in paragraph b) is higher than in one or more control biological samples.


SEQUENCE LISTING (4 sequences)

SEQ ID NO: 1 - DNA, 20 bp, Homo sapiens
cggctaacggataagcagag

SEQ ID NO: 2 - DNA, 20 bp, Homo sapiens
acctacagctggctcctgaa

SEQ ID NO: 3 - DNA, 20 bp, Homo sapiens
aaaagcccacatttgaggtg

SEQ ID NO: 4 - DNA, 20 bp, Homo sapiens
gaaattcctgaaaccgacca